(19) United States
(12) Patent Application Publication
(10) Pub. No.: US 2022/0250829 A1
(43) Pub. Date: Aug. 11, 2022

(54) Disinfectant Aerosol Fogger Apparatus

(71) Applicant: McKinley Brown, Columbia, SC (US)

(72) Inventor: McKinley Brown, Columbia, SC (US)

(21) Appl. No.: 17/168,356

(22) Filed: Feb. 5, 2021

(51) Int. Cl. B65D 83/28; B65D 83/14; B65D 83/44; A61L 2/26; A61L 2/22

(52) U.S. Cl. CPC B65D 83/28; B65D 83/753; B65D 83/44; A61L 2202/14; A61L 2/22; A61L 2202/15; A61L 2/26

(57) ABSTRACT

A disinfectant aerosol fogger apparatus for efficiently sanitizing an entire room includes a canister holding a compressed disinfectant agent. A valve stem has a stem bottom end coupled to a canister top side and a stem top end. The valve stem is in fluid communication with the canister. A valve is coupled within the valve stem to selectively block or allow release of the compressed disinfectant agent from the canister. The valve has a valve controller to manipulate the valve. A nozzle housing is coupled to the valve stem. The nozzle housing has a stem receiver to selectively receive the stem top end and a nozzle bar coupled to the stem receiver. The nozzle bar has a plurality of nozzles extending through a bar front side and a bar back side. Each nozzle has a nozzle aperture in fluid communication with the valve stem.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention.

The disclosure relates to disinfectant devices and more particularly pertains to a new disinfectant device for efficiently sanitizing an entire room.

### (2) Description of Related Art Including Information Disclosed Under 37 CFR 1.97 and 1.98.

The prior art relates to disinfectant devices. Known devices typically target a specific area, even if utilizing aerosol to create a spray. Those devices which create a “bomb” type effect release from a single nozzle. Most often this nozzle is oriented upwards and the devices like a plurality of horizontally oriented nozzles.

### BRIEF SUMMARY OF THE INVENTION

An embodiment of the disclosure meets the needs presented above by generally comprising a canister having a canister bottom side, a canister top side, and a canister sidewall extending therebetween. The canister holds a compressed disinfectant agent. A valve stem is coupled to the canister. The valve stem has a stem bottom end coupled to the canister top side and a stem top end. The valve stem is in fluid communication with the canister. A valve is coupled to the valve stem. The valve is coupled within the valve stem between the stem bottom end and the stem top end to selectively block or allow release of the compressed disinfectant agent from the canister. The valve has a valve controller to manipulate the valve. A nozzle housing is coupled to the valve stem. The nozzle housing has a stem receiver to selectively receive the stem top end and a nozzle bar coupled to the stem receiver. The nozzle bar has a plurality of nozzles extending through a bar front side and a bar back side. Each nozzle has a nozzle aperture in fluid communication with the valve stem.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of a disinfectant aerosol fogger apparatus according to an embodiment of the disclosure.

FIG. 2 is a front elevation view of an embodiment of the disclosure.

FIG. 3 is a side elevation view of an embodiment of the disclosure.

FIG. 4 is a rear elevation view of an embodiment of the disclosure.

FIG. 5 is a cross-sectional view of an embodiment of the disclosure along the line 5-5 of FIG. 1.

### DETAILED DESCRIPTION OF THE INVENTION

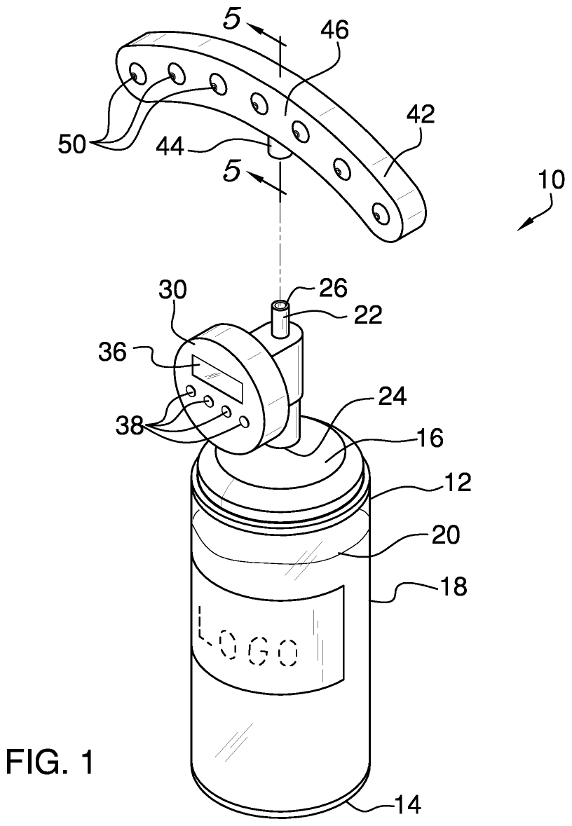

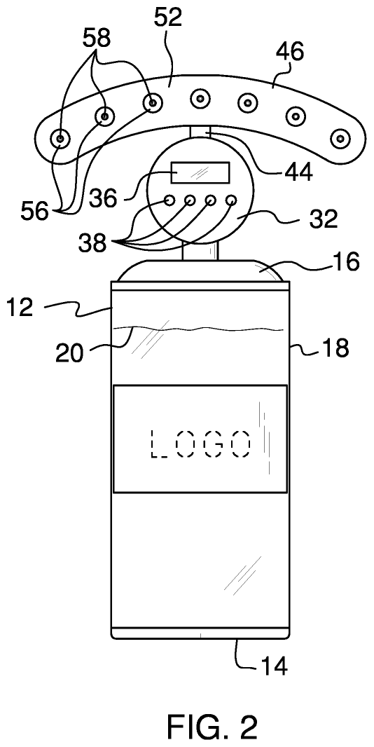

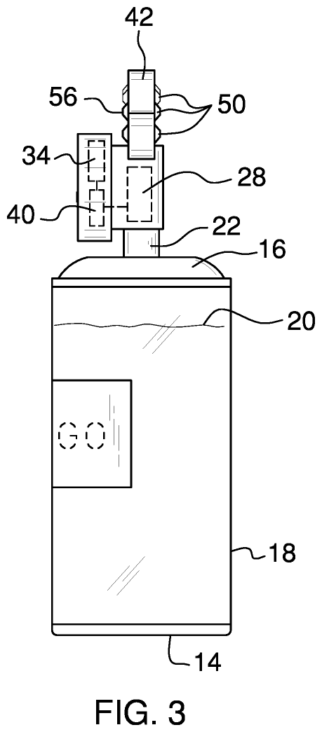

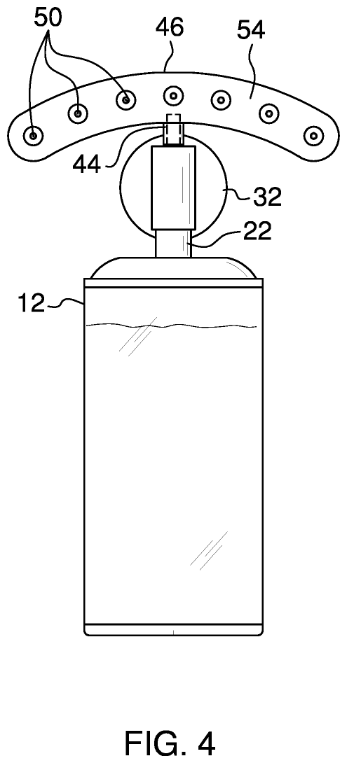

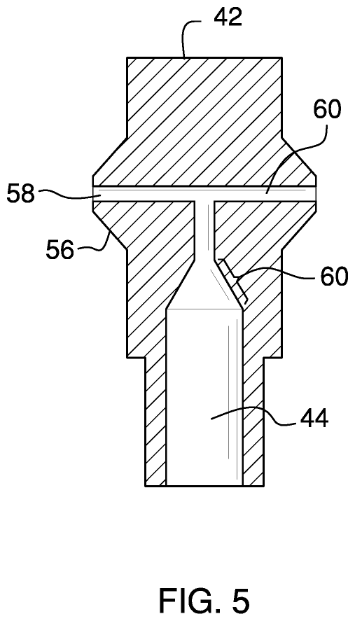

With reference now to the drawings, and in particular to FIGS. 1 through 5 thereof, a new disinfectant device embodying the principles and concepts of an embodiment of the disclosure and generally designated by the reference numeral 10 will be described.

As best illustrated in FIGS. 1 through 5, the disinfectant aerosol fogger apparatus 10 generally comprises a canister 12 having a canister bottom side 14, a canister top side 16, and a canister sidewall 18 extending therebetween. The canister 12 holds a compressed disinfectant agent 20. The disinfectant agent 20 may comprise at least 90% alcohol. The canister sidewall 18 may be transparent to allow the user to see the remaining amount of the disinfectant agent 20.

A valve stem 22 is coupled to the canister 12. The valve stem 22 has a stem bottom end 24 coupled to the canister top side 16 and a stem top end 26. The valve stem 22 is in fluid communication with the canister 12. A valve 28 is coupled within the valve stem 22 between the stem bottom end 24 and the stem top end 26 to selectively block or allow release of the compressed disinfectant agent 20 from the canister 12.

The valve 28 has a valve controller 30 to manipulate the valve 28. The valve controller 28 may include a timer housing 32 coupled to the valve stem 22. A timer 34 is coupled within the timer housing 32 and is in operational communication with the valve 28 to create a timed release of the disinfectant agent 20 in order for the user to have time to safely exit the room. The timer housing 32 may be circular and may have a display screen 36 and a plurality of control buttons 38 in operational communication with the timer 34. The timer 34 has a battery 40.

A nozzle housing 42 is coupled to the valve stem 22. The nozzle housing 42 has a stem receiver 44 to selectively receive the stem top end 26 and a nozzle bar 46 coupled to the stem receiver 44. The stem receiver 44 may engage the valve stem 22 with a press fit to allow for secure but easy changes once the canister 12 no longer contains the disinfectant agent 20.

The nozzle bar 46 may be arcuate and has a pair of rounded distal ends 48. The nozzle bar 46 has a plurality of nozzles 50 extending through a bar front side 52 and a bar back side 54. Each nozzle 50 may have a conical protrusion 56 and a nozzle aperture 58 in fluid communication with the valve stem 22 via the stem receiver 44. The stem receiver 44 may be cylindrical and may have a tapered portion 60 connecting to a feeder tube 62 that extends to the nozzle aperture 58 of each of the plurality of nozzles. The plurality of nozzles 50 may include seven nozzles 50 extending through the bar front side 52 and seven nozzles 50 extending through the bar back side 54.

In use, the user places the canister 12 in a room in need of disinfection. The user then manipulates the timer 34 and the valve controller 28 to start a delayed release of the disinfectant agent 20 from the plurality of nozzles 50.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A disinfectant aerosol fogger apparatus comprising:
a canister having a canister bottom side, a canister top side, and a canister sidewall extending therebetween, the canister holding a compressed disinfectant agent;
a valve stem coupled to the canister, the valve stem having a stem bottom end coupled to the canister top side and a stem top end, the valve stem being in fluid communication with the canister;
a valve coupled to the valve stem, the valve being coupled within the valve stem between the stem bottom end and the stem top end to selectively block or allow release of the compressed disinfectant agent from the canister, the valve having a valve controller to manipulate the valve; and
a nozzle housing coupled to the valve stem, the nozzle housing having a stem receiver to selectively receive the stem top end and a nozzle bar coupled to the stem receiver, the nozzle bar having a plurality of nozzles extending through a bar front side and a bar back side, each nozzle having a nozzle aperture in fluid communication with the valve stem.

2. The disinfectant aerosol fogger apparatus of claim 1 further comprising the nozzle bar being arcuate.

3. The disinfectant aerosol fogger apparatus of claim 1 further comprising the nozzle bar having a pair of rounded distal ends.

4. The disinfectant aerosol fogger apparatus of claim 1 further comprising each nozzle having a conical protrusion.

5. The disinfectant aerosol fogger apparatus of claim 1 further comprising the plurality of nozzles including seven nozzles extending through the bar front side and seven nozzles extending through the bar back side.

6. The disinfectant aerosol fogger apparatus of claim 1 further comprising the stem receiver being cylindrical and having a tapered portion connecting to a feeder tube extending to the nozzle aperture of each of the plurality of nozzles.

7. The disinfectant aerosol fogger apparatus of claim 1 further comprising the valve controller including a timer housing coupled to the valve stem, a timer being coupled within the timer housing to create a timed release.

8. The disinfectant aerosol fogger apparatus of claim 7 further comprising the timer housing being circular and having a display screen and a plurality of control buttons.

9. The disinfectant aerosol fogger apparatus of claim 1 further comprising the disinfectant agent comprising at least 90% alcohol.

10. A disinfectant aerosol fogger apparatus comprising:
a canister having a canister bottom side, a canister top side, and a canister sidewall extending therebetween, the canister holding a compressed disinfectant agent, the disinfectant agent comprising at least 90% alcohol;
a valve stem coupled to the canister, the valve stem having a stem bottom end coupled to the canister top side and a stem top end, the valve stem being in fluid communication with the canister;
a valve coupled to the valve stem, the valve being coupled within the valve stem between the stem bottom end and the stem top end to selectively block or allow release of the compressed disinfectant agent from the canister, the valve having a valve controller to manipulate the valve, the valve controller including a timer housing coupled to the valve stem, a timer being coupled within the timer housing to create a timed release, the timer housing being circular and having a display screen and a plurality of control buttons; and
a nozzle housing coupled to the valve stem, the nozzle housing having a stem receiver to selectively receive the stem top end and a nozzle bar coupled to the stem receiver, the nozzle bar being arcuate and having a pair of rounded distal ends, the nozzle bar having a plurality of nozzles extending through a bar front side and a bar back side, each nozzle having a conical protrusion and a nozzle aperture in fluid communication with the valve stem, the stem receiver being cylindrical and having a tapered portion connecting to a feeder tube extending to the nozzle aperture of each of the plurality of nozzles, the plurality of nozzles including seven nozzles extending through the bar front side and seven nozzles extending through the bar back side.
